(19) United States
(12) Patent Application Publication
(10) Pub. No.: US 2026/0117155 A1
(43) Pub. Date: Apr. 30, 2026

(54) APPARATUS, SYSTEM AND METHOD FOR MOUNTING A DEVICE TO A BIOPROCESING CONTAINER

(71) Applicants: Pall Life Sciences Belgium BV, Hoegaarden (BE); Global Life Sciences Solutions USA LLC, Marlborough, MA (US)

(72) Inventors: Alan Yim, Marlborough, MA (US); Nishad Pore, Marlborough, MA (US); Steven van den Berghe, Hoegaarden (BE)

(73) Assignees: Pall Life Sciences Belgium BV, Hoegaarden (BE); Global Life Sciences Solutions USA LLC, Marlborough, MA (US)

(21) Appl. No.: 18/928,346

(22) Filed: Oct. 28, 2024

(51) Int. Cl. C12M 1/00; C12M 3/00

(52) U.S. Cl. CPC C12M 23/14; C12M 23/26; C12M 23/28; C12M 23/46; C12M 23/50

(57) ABSTRACT

An attachment system and associated methods for attaching a device, such as a camera, to a flexible bag. The attachment system includes a frame and clamp that lock the device to the bag such that the camera remains fixed to the bag when the bag is in use, such as during bioprocessing operations. The clamp can lock and unlock the device from the bag through one-handed operation.

### TECHNICAL FIELD

Embodiments of the invention relate generally systems and methods for attaching a device to a bioprocessing container, and in particular, attachment systems and methods for attaching an imaging device to a flexible bioprocessing bag.

### BACKGROUND

Bioreactors and mixers are often employed to carry out biochemical and/or biological processes and/or manipulate liquids and other products of such processes. Such bioreactors and mixers often include flexible or collapsible single-use disposable bags that are supported by an outer rigid structure such as a stainless-steel shell or frame. The bags are made of thin flexible sheets of plastic film and are positioned within the rigid shell and filled with the desired fluid for processing.

Certain processes carried out in said bioreactors and mixers, such as growing biological materials such as mammalian cells, bacteria or yeast in a bioreactor often results in the production of an unwanted foam layer which floats at the top of the fluid in the bioreactor, e.g., in a headspace of a bioreactor bag. This foam layer is the result of several factors including the addition of pressurized air to sustain aerobic microorganisms, nutrients and growth factors present in the liquid growth media, and waste products generated by the microorganisms, Over time, this foam layer may become unacceptably thick, and, if untreated, could potentially foul the exhaust port and filter of a bioreactor, prevent CO₂ from escaping, and negatively affect the structural integrity of the bag. Foam also forms a barrier to liquids injected from above the fluid in the bioreactor and is problematic even at low fluid volume levels.

To reduce the foam layer, chemical solutions such as antifoam compounds are typically employed. With respect to such compounds, several applications may be needed during a single production run to ensure effectiveness. Conversely, too much antifoam compound can be detrimental to the biological materials in the reactor. Mechanical solutions, such as thermal probes and foam breakers also exist, however, they are more effective at reducing substantial amounts of existing foam rather than inhibiting foam formation.

Thus, accurate detection and monitoring of foam in a bioreactor bag is important to determine when intervention is necessary. One way of detecting foam is via imaging by way of a camera. However, for a camera to provide accurate data, a consistent field of view is required. This can be particularly challenging in bioprocessing applications, especially where the container is a flexible bag, and thus does not have a rigid, planar surface to mount the camera to. Moreover, the flexible bag, in use, is filled with a liquid and may be aerated (e.g., via sparging) and/or undergo mixing operations (e.g., via an impeller). Each of these operations cause the bag to move during use, making it even more difficult to mount the camera to the flexible bag in a way that allows the camera to reliably view the contents of the bag with a consistent field of view.

To have a wide field of view, the camera should be generally mounted at the center of the top of the bag. For large-scale manufacturing, such bags are very large and accessing the top, center of the bag can be difficult. Thus, any mechanism for attaching or otherwise fixing the camera to the bag needs to be easy to use, ideally with minimal operator intervention.

Other bioprocessing operations may require the use of physical equipment that needs to be attached to the flexible bag in a specific orientation, such as optical probes and the like. In these situations, the above concerns are similarly present, and there is a similar need for mechanism for attaching or otherwise fixing such devices to the flexible bag,

In view of the above, there is a need for an apparatus and system attaching a device, such as a camera, to a flexible bag in a consistent and repeatable manner.

### BRIEF DESCRIPTION

Certain embodiments commensurate in scope with the originally claimed subject matter are summarized below. These embodiments are not intended to limit the scope of the claimed subject matter, but rather these embodiments are intended only to provide a summary of the possible embodiments. Indeed, the disclosure may encompass a variety of forms that may be like or different from the embodiments set forth below.

According to a first aspect, a clamp (200) configured to attach a device (400) to a flexible bag (300) is provided, comprising: a baseplate (202); a first locking rod (210) having a proximal end (211) and a distal end (212); a second locking rod (210) having a proximal end (211) and a distal end (212); a handle (220) connected to the proximal end (211) or distal end (212) of the first and second locking rods (210); wherein the first and second locking rods (210) are each configured to slide within a respective channel (204) in the baseplate (202); and wherein sliding the handle (220) in a first direction (100) causes the first and second locking rods to slide within their respective channels (204) in the first direction and lock the device to the flexible bag (300).

The clamp may further include at least one adapter (230) connected to or integral with the baseplate (202), wherein the adapter (230) is configured to be attached to the device (400).

The flexible bag (300) comprises at least one port (310) and the baseplate comprises an opening (206), and wherein the port (310) is configured to fit inside the opening (206). The port (310) comprises a top flange (316) and a bottom flange (318), creating a recess (320) therebetween, wherein the first and second locking rods (210) each have a cutout (213) between the proximal end (211) and distal end (212) thereof, and wherein sliding the handle (220) in the first direction (100) moves the cutouts (213) from being located in the recess (320) to being located outside of the recess (320). When the cutout (213) of the first and second locking rods (210) are located in the recess, the clamp (200) is not attached to the flexible bag, and when the handle (220) is slid in the first direction (100), an outer surface (214) of the first and second locking rods (210) is pressed against the port (310), thereby attaching the clamp (200) to the flexible bag (300).

According to a second aspect, a bioprocessing system is provided, comprising: a clamp (200) configured to attach a device (400) to a flexible bag (300) is provided, comprising: a baseplate (202); a first locking rod (210) having a proximal end (211) and a distal end (212); a second locking rod (210) having a proximal end (211) and a distal end (212); a handle (220) connected to the proximal end (211) or distal end (212) of the first and second locking rods (210); wherein the first and second locking rods (210) are each configured to slide within a respective channel (204) in the baseplate (202); and wherein sliding the handle (220) in a first direction (100) causes the first and second locking rods to slide within their respective channels (204) in the first direction and lock the device to the flexible bag (300), the device (400) attached to the clamp (200); and the flexible bag (300), wherein the device is a camera, and wherein the flexible bag (300) is a single-use bag (300) configured to carry out a bioprocessing operation.

According to a third aspect, an attachment system (500) for attaching a device (400) to a flexible bag (300) is provided, comprising: a frame (501) attached to a vessel that bounds the flexible bag (300), comprising: one or more support legs (510) attached to the vessel; one or more cross bars (520) connected to at least one of the one or more support legs (510); a sliding arm (530), slidably engaged with one of the one or more cross bars (520); and a mount (560) moveably engaged with the sliding arm (530), and a clamp (200), attached to the mount (560), comprising: a baseplate (202); a first locking rod (210) having a proximal end (211) and a distal end (212); a second locking rod (210) having a proximal end (211) and a distal end (212); a handle (220) connected to the proximal end (211) or distal end (212) of the first and second locking rods; wherein the first and second locking rods (210) are each configured to slide within a respective channel (204) in the baseplate (202); and wherein sliding the handle (220) in a first direction (100) causes the first and second locking rods to slide within their respective channels (204) in the first direction and lock the device (400) to the flexible bag (300).

The sliding arm (530) is configured to slide along the cross bar (520) in a first plane, and wherein the mount (560) is configured to slide along the sliding arm in a second plane that is perpendicular to the first plane.

The device (400) is attached to the mount (560) and configured to be aligned with a port (310) of the bag (300), wherein sliding the handle (220) in the first direction (100) locks the clamp (200) to the port (310). The sliding arm (530) further comprises a rod (550), and wherein the mount (560) is configured to rotate about a longitudinal axis of the rod (550). The sliding arm (530) further comprises a first latch (540), wherein the mount (560) further comprises a second latch (570), and wherein the first and second latch (540, 570) are configured to engage each other such that the mount (560) is locked in a raised position.

In embodiments, the device (400) is a camera, and wherein the attachment system (100) is configured to allow the camera (400) to move relative to the frame (500) and/or vessel when locked to the flexible bag.

According to a fourth aspect, A method of attaching a device (400) to a flexible bag (300) is provided, the method comprising: attaching the device (400) to clamp (200), wherein the clamp comprises: a baseplate (202); a first locking rod (210) having a proximal end (211) and a distal end (212); a second locking rod (210) having a proximal end (211) and a distal end (212); and a handle (220) connected to the proximal end (211) or distal end (212) of the first and second locking rods; wherein the first and second locking rods (210) are each configured to slide within a respective channel (204) in the baseplate (202), and sliding the handle (220) in a first direction (100) such that the first and second locking rods slide within their respective channels (204) in the first direction to lock the device (400) to the port (310) of the flexible bag (300).

The clamp (200) is connected to a mount (560) moveably engaged with a frame (501), the frame (501) attached to a vessel that bounds the flexible bag (300), the method further comprising: moving the mount (560) relative to the frame (501) and vessel, such that the device (400) and associated clamp (200) are aligned with a port (310) attached to the flexible bag (300).

In embodiments, the device (400) is a camera, wherein the flexible bag (300) is a single-use flexible bag for carrying out a bioprocess operation, and wherein sliding the handle (220) in the first direction occurs after the single-use flexible bag (300) is inflated within the vessel.

In embodiments, moving the mount (560) relative to the frame (501) and vessel, such that the device (400) and associated clamp (200) are aligned with a port (310) attached to the flexible bag (300), comprises: sliding a sliding arm (530) of the frame (500) along a cross bar (520); unlatching the mount (560) from the sliding (arm); and lowering the mount (560), relative to the sliding arm (530), such that an opening in the baseplate (202) is aligned with the port (310). Moving the mount (560) relative to the frame (501) and vessel further comprises rotating the mount (560) relative to the sliding arm (530). Sliding the handle (220) in a first direction (100) such that the first and second locking rods slide within their respective channels (204) in the first direction to lock the device (400) to the port (310) of the flexible bag (300) is carried out by a single-handed operation.

In embodiments, the method further comprises sliding the handle (220) in a second direction (101) to unlock the device (400) from the port (310).

In embodiments, the method further comprises using the camera (400) to image an interior of the flexible bag (300) during the bioprocessing operation. During the bioprocessing operation the flexible bag (300) moves relative to the vessel, and the device (400) moves in response to movement of the flexible bag (300), such that the device (400) maintains alignment with the port (310) during the bioprocessing operation.

### DRAWINGS

The present invention will be better understood from reading the following description of non-limiting embodiments, with reference to the attached drawings, wherein below:

FIGS. 1 and 2 illustrate perspective views of a clamp in an unlocked and locked state, respectively, according to embodiments of the invention.

FIGS. 3 and 4 illustrate top views of the clamp of FIGS. 1 and 2 in an unlocked and locked state, respectively, according to embodiments of the invention.

FIGS. 5 and 6 illustrate bottom views of the clamp of FIGS. 1 and 2 in a locked and unlocked state, respectively, according to embodiments of the invention.

FIGS. 7 and 8 illustrate perspective views of the clamp of FIGS. 1 and 2 with an associated flexible bag in an unlocked and locked state, respectively, according to embodiments of the invention.

FIGS. 9 and 10 illustrate perspective views of the clamp of FIGS. 1 and 2 having a device attached thereto in an unlocked and locked state, respectively, according to embodiments of the invention.

FIGS. 11 and 12 illustrate perspective views of the clamp of FIGS. 1 and 2 having a device attached thereto in an unlocked and locked state, respectively, according to embodiments of the invention, with a baseplate of the clamp being transparent.

FIGS. 13 and 14 illustrate top views of the clamp of FIGS. 1 and 2 having a device attached thereto in an unlocked and locked state, respectively, according to embodiments of the invention, with a baseplate of the clamp being transparent.

FIG. 15 illustrates a locking rod of the clamp of FIGS. 1 and 2 according to embodiments of the invention.

FIGS. 16 and 17 provide cut-through side views along a center line of the clamp of FIGS. 1 and 2 with the clamp aligned with a port of a flexible bag.

FIG. 18 illustrates a top view of the clamp of FIGS. 1 and 2, with the device rotated at an angle relative to the baseplate.

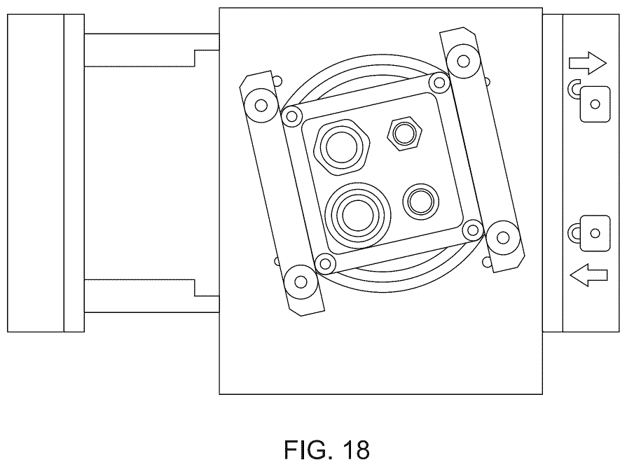

FIG. 19 illustrates a front perspective view of the clamp of FIGS. 1 and 2 attached to a frame for aligning the device with a port of a flexible bag.

FIG. 20 illustrates a back perspective view of the clamp of FIGS. 1 and 2 attached to a frame for aligning the device with a port of a flexible bag.

FIG. 21 illustrates a front perspective view of the clamp of FIGS. 1 and 2 attached to a frame and aligned with a port of a flexible bag.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor or mixer vessels having a wall or a portion of a wall that is flexible or semi-rigid, single use flexible bags, as well as other containers or conduits commonly used in biological or biochemical processing, including, for example, cell culture/purification systems, fermentation systems, mixing systems, media/buffer preparation systems, and filtration/purification systems.

As used herein, the term “bag” means a flexible or semi-rigid container or vessel used, for example, as a bioreactor or mixer for the contents within. While embodiments of the present invention are described as for use with bioprocessing bags, including but not limited to bioreactor bags and mixer bags, embodiments may also be configured for use with other bags or vessels. Similarly, embodiments may be used to image, assess, and mitigate/treat other characteristics or conditions, in addition to the accumulation of foam in a bioreactor.

Further, while embodiments are described in connection with single use, stirred tank bioreactors, mixers, bioreactor systems, and mixing systems, they are not limited to the same and may be used with a variety of vessels and associated equipment used in biological or biochemical processing. Additionally, embodiments may be suitable for use in other devices and to other types of biocontainers.

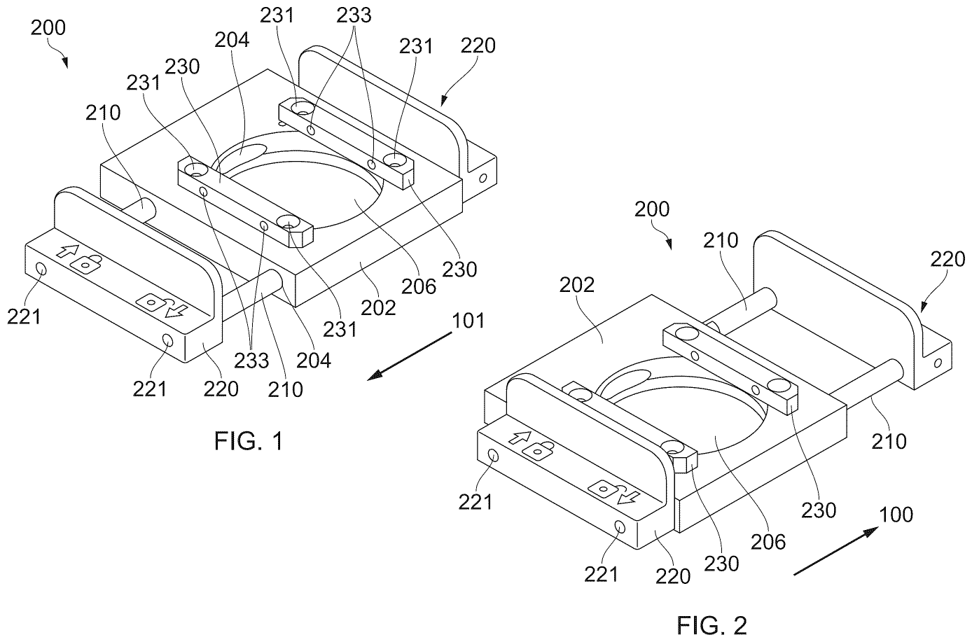

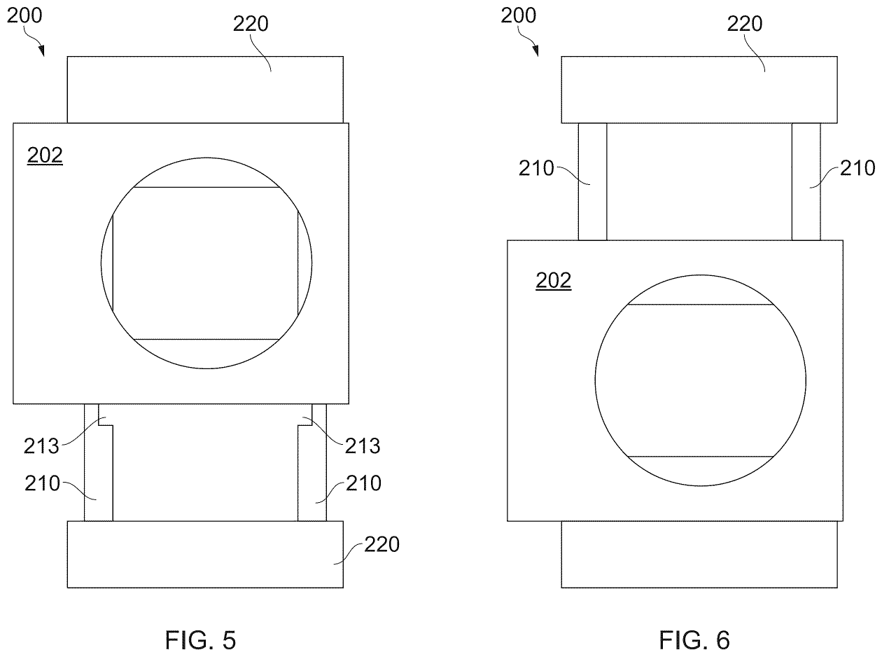

According to a first aspect, and with reference to FIGS. 1 and 2, a clamp 200 is illustrated in an unlocked and locked orientation, respectively. The clamp 200 includes baseplate 202 having a generally centrally located opening 206 therein. First and second locking rods 210 are located in channels 204 along opposite sides of the baseplate 202 and are configured to slide within said channels 204. A first handle 220 is attached to proximal ends 211 of the locking rods 210, and a second handle 220 is attached to distal ends 212 of the locking rods 210. The handles 220 are connected or otherwise attached to the locking rods 210. In one example, locking rods 210 each have a threaded hole 215 on their proximal and distal surfaces. Handles 220 also have a hole (threaded or unthreaded) 221 that is aligned with the threaded hole 215. A screw (not shown) or other threaded engagement element can be pass through the hole 221 and engage with hole 215, such that the handles 220 are connected to the locking rods 210. In other embodiments, the handles 220 and locking rods 210 are connected by any other suitable means, such as via welding, gluing, snap fit, etc. As FIGS. 1-12 illustrate, the handles 220 and locking rods 210 are designed such that they can slide back and forth in first direction 100 and second direction 101 to lock and unlock the clamp from a port 310.

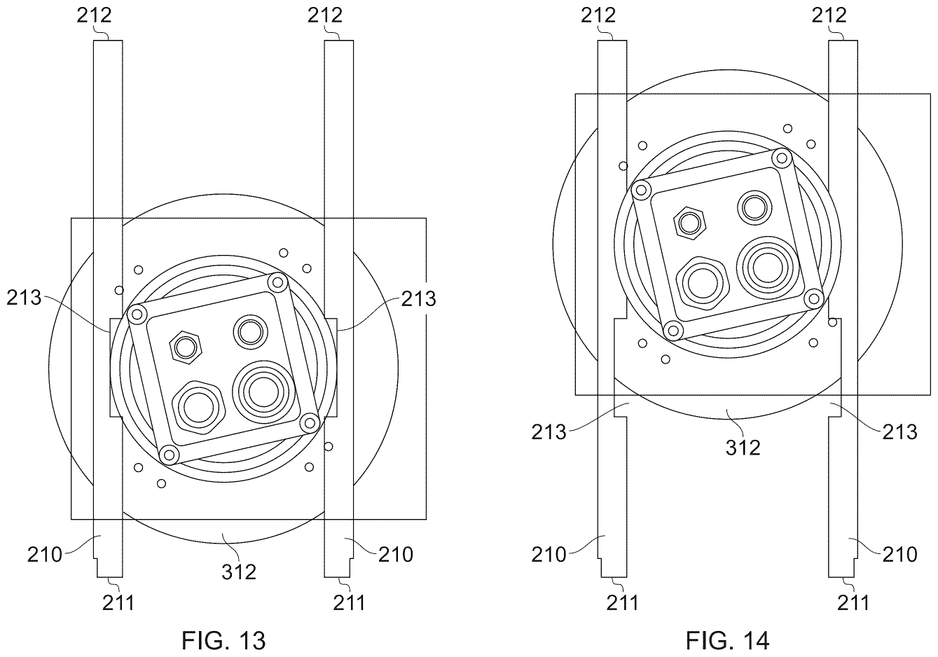

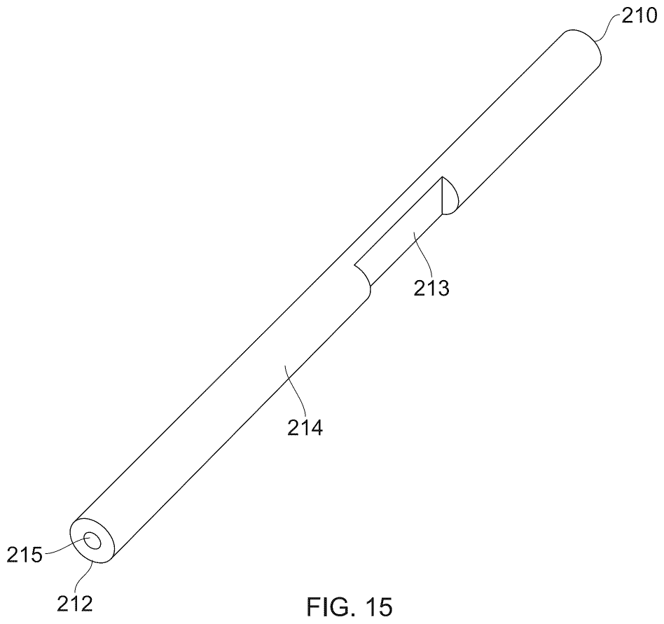

The clamp 200 is designed such that the locking rods 210 slide within the channels 204 of baseplate 202 such that cutouts 213 in rods 210 (see e.g., FIG. 15) can be located within or past opening 206 (see e.g., FIGS. 13-14), the amount of movement being limited by the handles 220 abutting the baseplate 202.

Baseplate 202 may include one or more adapters 230, which are configured to attach a device 400 to the baseplate 202. As illustrated in FIGS. 1-14, first and second adaptors 230 are attached, integral with, or otherwise fixed to the baseplate 202 near the opening 206. In one example, the adaptors 230 are attached to the baseplate 202 through use of a threaded engagement element (e.g., threaded screw). Specifically, each adaptor 230 includes one or more holes 231 that are aligned with threaded holes (not shown) in baseplate 202. When aligned threaded engagement element (e.g., threaded screw) can be threaded through the hole 231 and into the threaded holes in the baseplate 202, thereby attaching the adaptor 230 to the base plate 202. In other embodiments, the adaptors 230 and baseplate 230 are connected by any other suitable means, such as via welding, gluing, snap fit, etc. The adaptors 230 include additional holes 233 that are configured to align with holes 440 in the body 410 of device 400. Like above, the device 400 can be attached to the adapters 230 via threaded engagement elements (e.g., screws). In other embodiments, the adaptors 230 and device 400 are connected by any other suitable means, such as via welding, gluing, snap fit, etc.

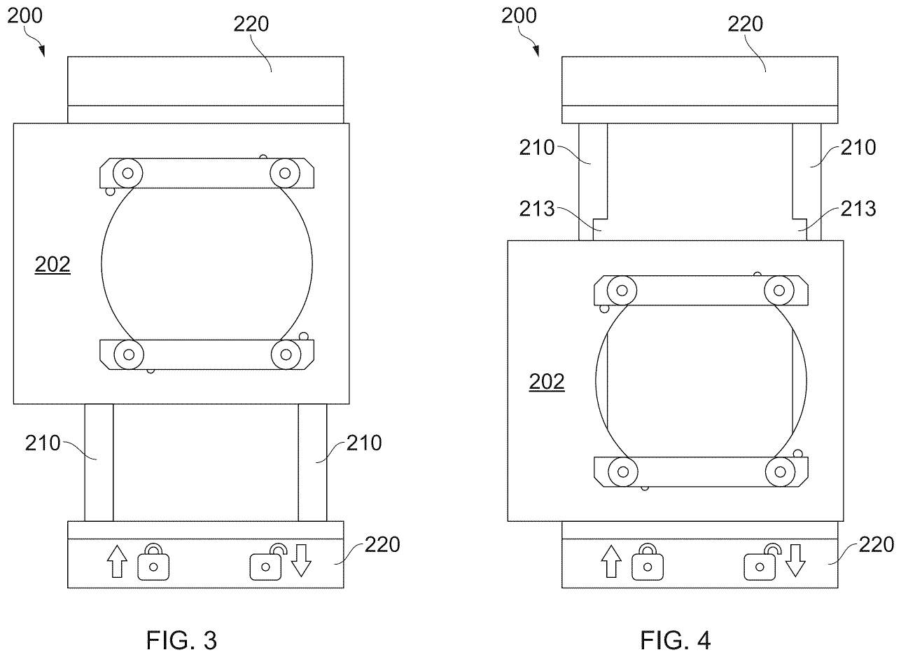

FIGS. 3-4 illustrate tops view of the clamp 200 of FIGS. 1-2, and FIGS. 5 and 6 illustrate bottom views of the claim of FIGS. 1-2, in unlocked and locked states. As these figures show, and as will be explained in greater detail below, in the unlocked position the opening 206 is unobstructed because cutouts 213 in the locking rods 210 are located in portions of the channels 204 that overlap with opening 206 (see, e.g., FIGS. 3, 6, 11 and 13). Conversely, in the locked position the opening 206 is partially obstructed by the outer surface 214 of locking rods 210 (see, e.g., FIGS. 4, 5, 8, 12, and 14), with the cutouts 213 being located in a portion of channels 204 that is past the opening 206.

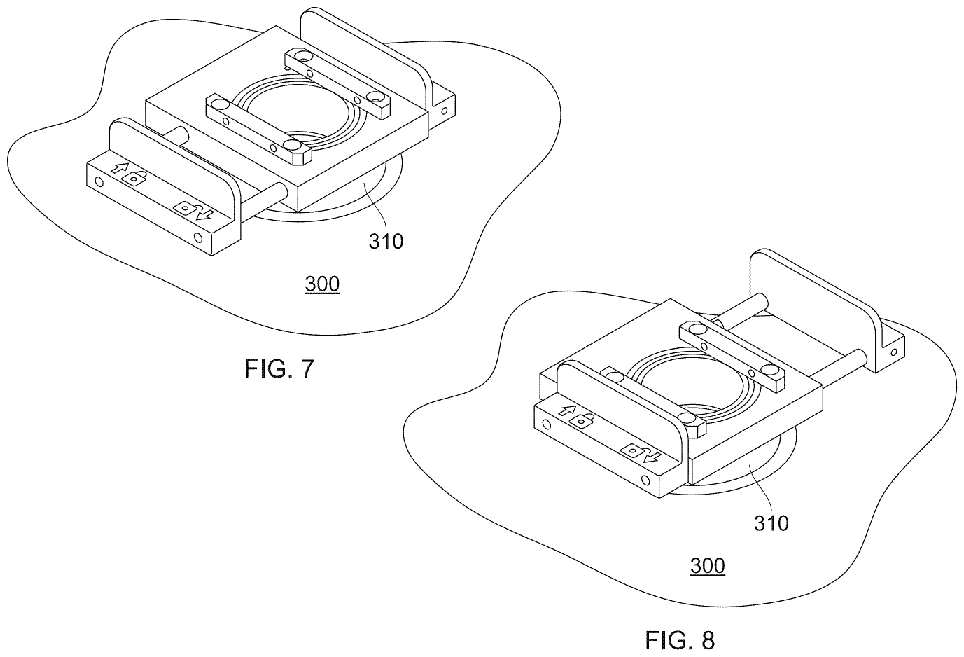

As mentioned in the background, there is a need for a mechanism to attach a device 400, such as a camera, to surface of a flexible bag 300, where the flexible bag is inflated and configured to carry out a bioprocessing operation. As FIGS. 7-17 illustrate, a flexible bag 300 for carrying out a bioprocessing operation includes a port 310 protruding therefrom. The opening 206 is sized to have a shape larger than, but generally conforming to, the shape of the port 310, such that the port 310 can fit within the opening 206 (see, e.g., FIGS. 7 and 8).

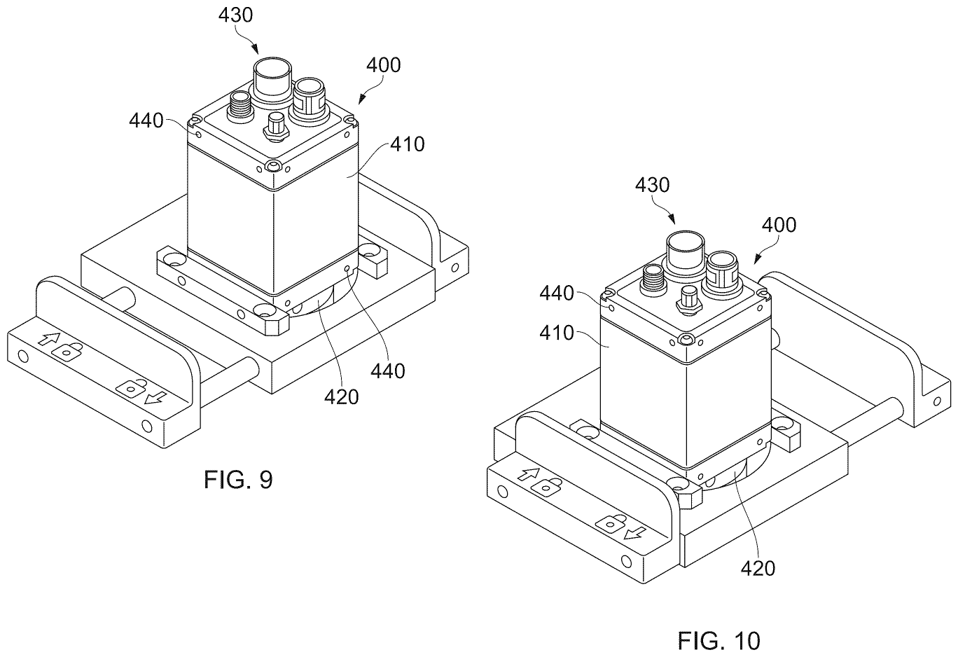

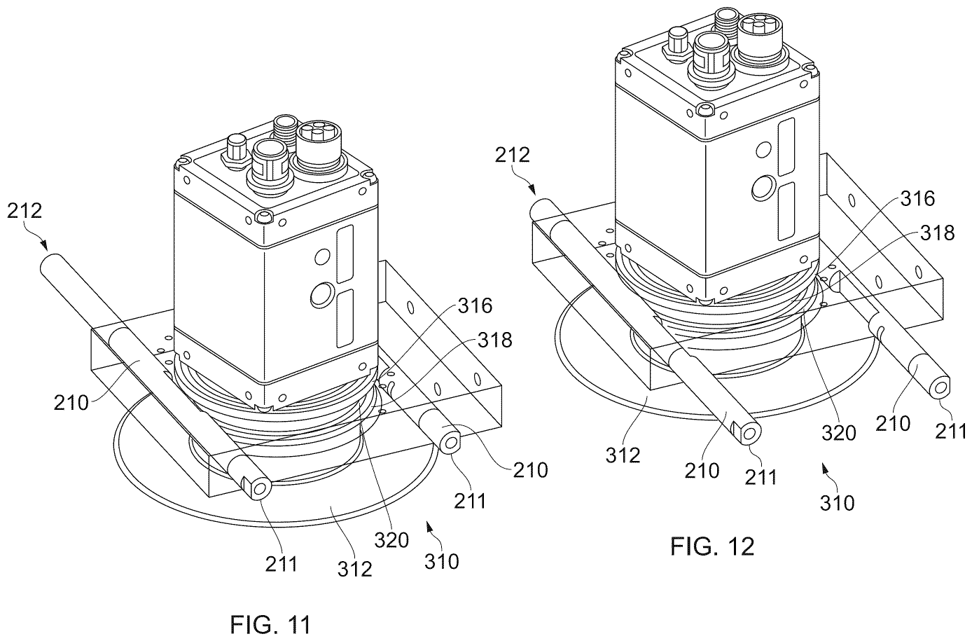

The device 400, as described above, and best shown in FIGS. 9 and 10, can be attached to the clamp 200 such that the device 400 is fixed above and/or within the opening 206. In embodiments where the device is a camera 400 having a lens 420, the lens 420 can protrude into opening 206, such that it can sit within the port 310 (see, e.g., FIGS. 11-16). Additionally the back of the device 400 may include electrical connections 430 that are configured to connect to suitable cables or wires.

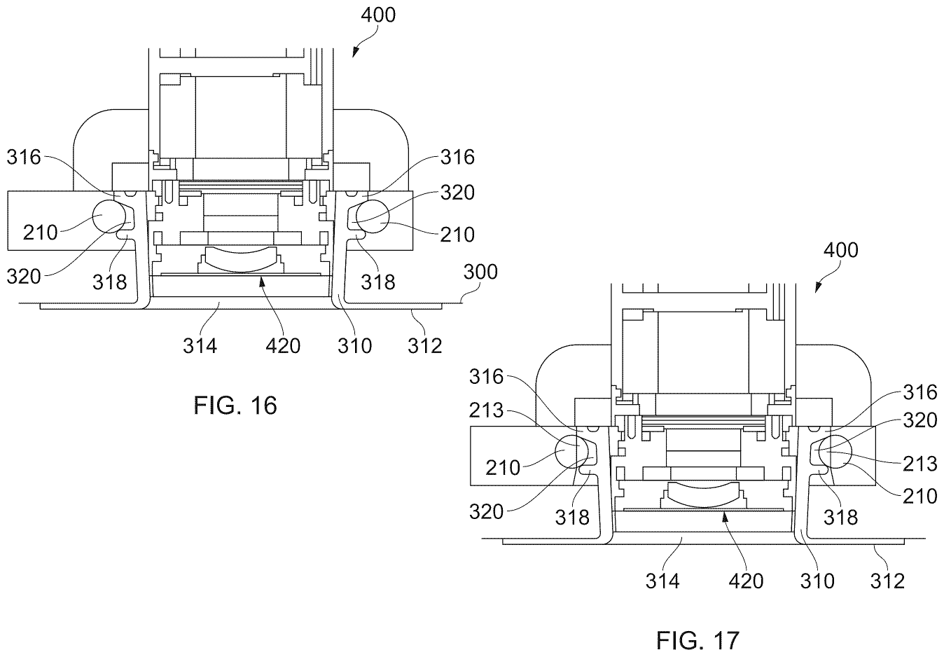

The port 310, which may be a standard port fitting used in the bioprocessing industry, includes a top flange 316 and a bottom flange 318, creating a recess 320 therebetween (best seen in FIGS. 11, 12, 16, and 17). A base 312 of the port is welded to the bag 300 and includes an optically transparent window 314 in its center. In use, the clamp 200 (with the device 400 already attached) is placed over the port 310, such that the port 310 sits within the opening 206. As best shown in FIGS. 16 and 17, when the device 400 is a camera, the lens 420 is configured to sit within the port 310 such that the lens 420 is close to the surface of the flexible bag 300, thereby ensuring the camera has a good field of view of the contents of the bag 300.

As briefly mentioned above, the clamp starts in the unlocked state, such that the cutouts 213 in the locking rods 210 are located in portions of the channels 204 that overlap with opening 206 (see, FIGS. 11 and 13). Sliding the handle(s) 220 in the first direction 100 moves the cutouts 213 out of the overlapping area with the opening 206 such that an outer surface 214 of the locking rods is located within opening 216 (see, FIGS. 12, 14, and 16). As best shown in FIGS. 11-17, with specific reference FIGS. 16 and 17, when the cutouts 213 are located in the opening 206, the locking rods 210 do not abut top and bottom flanges 316, 318 of the port 310 (see FIG. 17), thereby allowing the clamp 200 to freely move relative to the port 310. When the handle(s) 220 are moved in the second direction 101, the cutouts 213 move past the opening 216, such that an outer surface 214 (See FIG. 15) of the locking rods is located within recess 320 and presses against top and bottom flanges 316, 318, locking or attaching the clamp 200 (and thereby the device 400) to the port (and thus the bag 300). In the locked position, the lens 420 is close in proximity to the window 314, thereby providing an improved field of view of the contents of the bag 300.

As alluded to above, the clamp 200 locks the device 400 to the port 310, such that the clamp 200 and device 400 cannot be easily pulled off of or otherwise separate from the bag 300. Additionally, by attaching the clamp 200 to the port 310, the device 400 is free to adjust it's position as the bag 300 moves (e.g., during a bioprocessing operation such as cell culturing, mixing, etc.), while still being in close proximity to the window 314. For example, if the device 400 (or clamp 200) was directly attached or fixed to the bag 300, movements in the bag 300 would cause a misalignment with the window 314 of the port 310, preventing the device 400 from being able to properly see inside the bag 300.

FIG. 17 illustrates how the device 400 can be attached to the clamp 200 in other configurations. As shown, the device 400 need not be attached to the clamp 200 such that the body 410 and clamp are parallel to each other. Rather, by adjusting where the holes 231 on the adaptors 230 align with holes on the baseplate 202, the device 400 can be attached to the clamp 200 in a rotated configuration. Adjusting the rotation of the device 400 provides the benefit of allowing a user to ensure that the device is aligned with (e.g., has a field of view of) other components of the overall bioprocessing system.

According to a second aspect, a method of attaching the device 400 to flexible bag 300 is disclosed. The method includes attaching the device 400 to the clamp 200 (as described above and omitted here), aligning the clamp 200 over the port 310 of the bag 300, such that the port 310 is within the opening 206 of the baseplate 202 of the clamp 200, and sliding the handle(s) 220 in the first direction 100 to lock the clamp 200 and device 400 to the bag 300. The sliding operation can be accomplished via a one-handed operation (i.e., a single hand can slide the handle(s) in the first direction without the need for both hands to keep the clamp aligned).

Similarly, the clamp 200 and device 400 are unlocked from the bag 300 by sliding the handle(s) 202 in the second direction 101. This is particularly advantageous in large-scale operations when the flexible bag is configured to contain hundreds or thousands of liters of fluid (e.g., cell culture media), where access to the top of the bag 300 requires a user to be on a ladder and thus one hand may be unavailable (e.g., needed to stabilize the user on the ladder) to grab the clamp.

A further advantage of the presently described method is that the locking and unlocking operation is quick and easy, without requiring difficult/complex locking mechanisms (e.g., hooks, snaps, etc). Moreover, by fixing the clamp 200 to the port 310 (as opposed to the bag 300 itself), a rigid surface is utilized for engagement (as opposed to a flexible surface of the bag). This is particularly helpful in bioprocessing operations, where the flexible bag 300 is inflated prior to attachment of the clamp 200.

As discussed above, in embodiments, the device 400 is a camera. In such embodiments, the clamp 200 advantageously aligns the lens 420 of the camera with the port 310 window 314 even when the surface of the bag 300 moves or adjusts during operational use. As such, the method can further include using the camera to image an interior of the flexible bag during a bioprocessing operation, without a loss of field of view. Such imaging can be used to detect foam levels inside the bag 300.

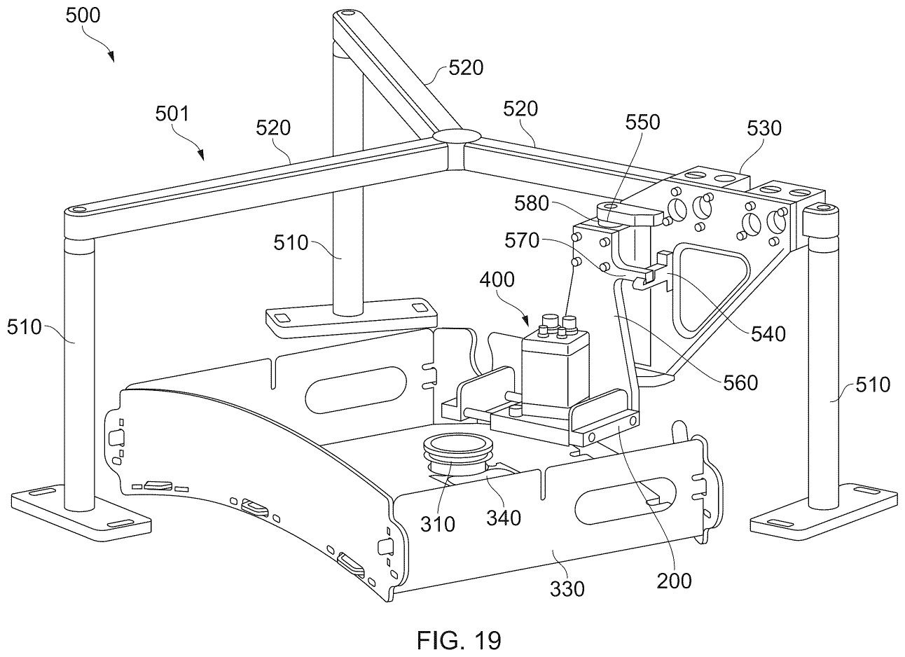

According to a further aspect, an attachment system 500 for attaching a device 400 to a flexible bag 300 is disclosed. The attachment system 500 includes a frame 501 and the previously described clamp 200. As illustrated in FIGS. 19-21, the frame 501, which is attached to a vessel (not shown) that bounds the flexible bag 300 in use, includes a one or more support legs 510 connected to the vessel, one or more cross bars 520, a sliding arm that is slidably engaged with one of the one or more cross bars 520, and a mount 560 that is movably engaged with the sliding arm 520. The clamp 200 (and associated attached device 400 in use), the details of which have been described above and are omitted here for brevity, are attached to the mount 560.

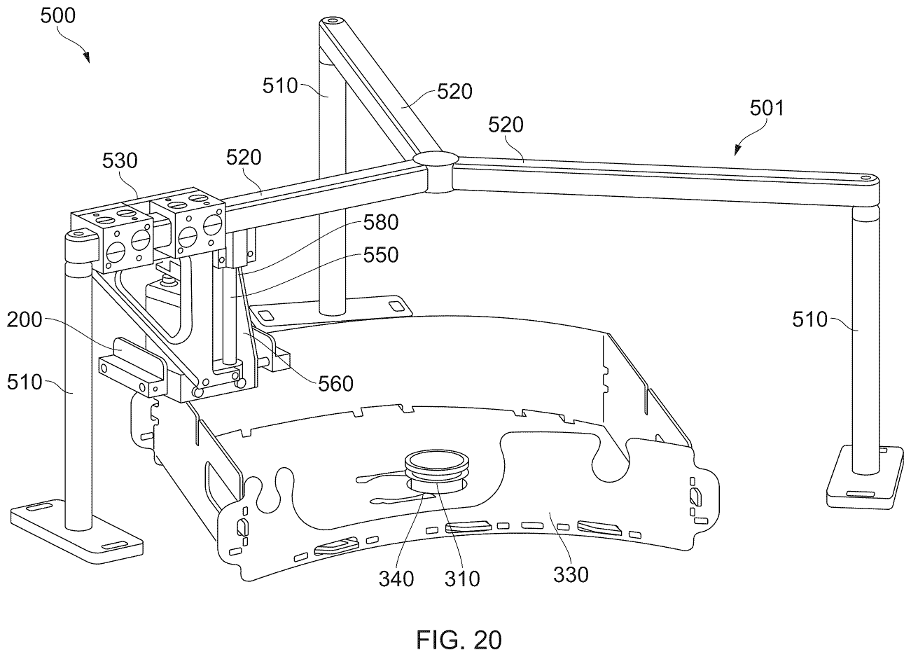

As best shown in FIGS. 19 and 20, prior to attaching the clamp 200 to the port 310, the attachment system 500 is configured such that clamp 200 and device 400 are off to the side so that the bag 300 can be installed in the vessel and inflated. In the examples shown in FIGS. 19-21, the bag 300 film is omitted, but the port 310 and an optional top tray 330 attached to the bag 300 are shown. The port 310 protrudes through a cutout 340 in the top tray such that the port 310 is accessible to a user.

As shown, at least one sliding arm 530 is slidably attached to a cross bar 520 and configured to slide along the cross bar 520 in a first plane. As best shown in FIG. 19, which is a front view of the attachment system 500, the sliding arm 530 includes a first latch 540 attached thereto. The mount 560 is also slidably attached to the sliding arm 530. As best shown in FIG. 20, which is a back view of the attachment system 500, the mount 560 includes a rod 550 that sits within a through hole 580 located on the back of the sliding arm 530 such that the mount can slide vertically along the rod 550. The mount also includes a second latch 570 configured to engage the first latch 560 prior to installation, such that the mount 560 is in a fully raised position (e.g., where top of the mount 560 abuts a surface of the sliding arm 530). This ensures that no components of the attachment system come into contact with the flexible bag 300 when aligning the clamp 200 with the port 310 or when inflating the flexible bag 300.

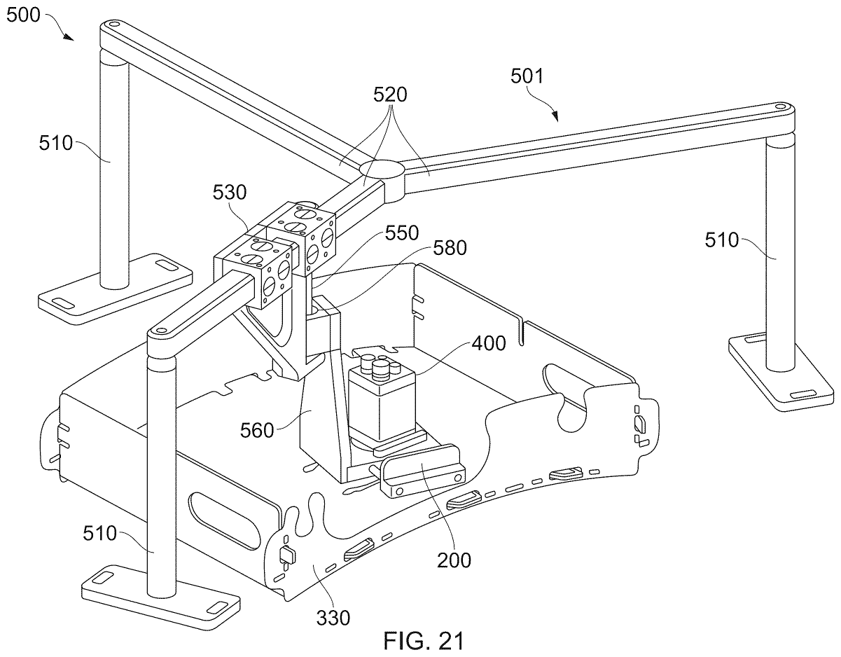

When a user is ready to attach the clamp 200 to the port 310, the user will slide the sliding arm 530 along its associated cross bar 520, disengage the first and second latches 540, 570, and lower the mount 560. By further sliding the sliding arm 530, lowering the mount 560 along rod 550, and rotating the mount 560 (around the longitudinal axis of the rod 550), the user can freely and easily align the clamp 200 with the port 310. Once aligned (see FIG. 21), the handle(s) 220 of the clamp 200 can be slid in the first direct to lock the clamp 200, and thus the device 400, to the port 310 and flexible bag 300.

As FIGS. 19-21 and the aforementioned description thereof provide, the attachment system 500 provides many degrees of freedom and movement operations, such that clamp 200 and device 400 can freely move relative to the bag 300, providing easy alignment and attachment thereof. Moreover, all operations to align the clamp 200 with the port 310 can be carried out through one-handed operation, which further eases installation.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A clamp configured to attach a device to a flexible bag, comprising:
a baseplate;
a first locking rod having a proximal end and a distal end;
a second locking rod having a proximal end and a distal end;
a handle connected to the proximal end or distal end of the first and second locking rods;
wherein the first and second locking rods are each configured to slide within a respective channel in the baseplate; and
wherein sliding the handle in a first direction causes the first and second locking rods to slide within their respective channels in the first direction and lock the device to the flexible bag.

2. The clamp of claim 1, further comprising:
at least one adapter connected to or integral with the baseplate, wherein the adapter is configured to be attached to the device.

3. The clamp of claim 1, wherein the flexible bag comprises at least one port and the baseplate comprises an opening, and
wherein the port is configured to fit inside the opening.

4. The clamp of claim 3, wherein the port comprises a top flange and a bottom flange, creating a recess therebetween,
wherein the first and second locking rods each have a cutout between the proximal end and distal end thereof, and
wherein sliding the handle in the first direction moves the cutouts from being located in the recess to being located outside of the recess.

5. The clamp of claim 4, wherein when the cutout of the first and second locking rods are located in the recess, the clamp is not attached to the flexible bag, and when the handle is slid in the first direction, an outer surface of the first and second locking rods is pressed against the port, thereby attaching the clamp to the flexible bag.

6. A bioprocessing system, comprising:
the clamp of claim 1;
a device attached to the clamp; and
a flexible bag,
wherein the device is a camera, and wherein the flexible bag is a single-use bag configured to carry out a bioprocessing operation.

7. An attachment system for attaching a device to a flexible bag, comprising:
a frame attached to a vessel that bounds the flexible bag, comprising:
one or more support legs attached to the vessel;
one or more cross bars connected to at least one of the one or more support legs;
a sliding arm, slidably engaged with one of the one or more cross bars; and
a mount moveably engaged with the sliding arm, and
a clamp attached to the mount, comprising:
a baseplate;
a first locking rod having a proximal end and a distal end;
a second locking rod having a proximal end and a distal end;
a handle connected to the proximal end or distal end of the first and second locking rods;
wherein the first and second locking rods are each configured to slide within a respective channel in the baseplate (202); and
wherein sliding the handle in a first direction causes the first and second locking rods to slide within their respective channels in the first direction and lock the device to the flexible bag.

8. The attachment system of claim 7, wherein the sliding arm is configured to slide along the cross bar in a first plane, and wherein the mount is configured to slide along the sliding arm in a second plane that is perpendicular to the first plane.

9. The attachment system of claim 7, further comprising the device, the device attached to the mount and configured to be aligned with a port of the bag, wherein sliding the handle in the first direction locks the clamp to the port.

10. The attachment system of claim 7, wherein the sliding arm further comprises a rod, and wherein the mount is configured to rotate about a longitudinal axis of the rod.

11. The attachment system of claim 7, wherein the sliding arm further comprises a first latch, wherein the mount further comprises a second latch, and wherein the first and second latch are configured to engage each other such that the mount is locked in a raised position.

12. The attachment system of claim 9, wherein the device is a camera, and wherein the attachment system is configured to allow the camera to move relative to the frame and/or vessel when locked to the flexible bag.

13. A method of attaching a device to a flexible bag, the method comprising:
attaching the device to clamp, wherein the clamp comprises:
a baseplate;
a first locking rod having a proximal end and a distal end;
a second locking rod having a proximal end and a distal end; and
a handle connected to the proximal end or distal end of the first and second locking rods; wherein the first and second locking rods are each configured to slide within a respective channel in the baseplate, and
sliding the handle in a first direction such that the first and second locking rods slide within their respective channels in the first direction to lock the device to the port of the flexible bag.

14. The method of claim 13, wherein the clamp is connected to a mount moveably engaged with a frame, the frame attached to a vessel that bounds the flexible bag, the method further comprising:
moving the mount relative to the frame and vessel, such that the device and associated clamp are aligned with a port attached to the flexible bag.

15. The method of claim 13, wherein the device is a camera, wherein the flexible bag is a single-use flexible bag for carrying out a bioprocess operation, and wherein sliding the handle in the first direction occurs after the single-use flexible bag is inflated within the vessel.

16. The method of claim 14, wherein moving the mount relative to the frame and vessel, such that the device and associated clamp are aligned with a port attached to the flexible bag, comprises:
sliding a sliding arm of the frame along a cross bar;
unlatching the mount from the sliding (arm); and
lowering the mount, relative to the sliding arm, such that an opening in the baseplate is aligned with the port.

17. The method of claim 16, wherein moving the mount relative to the frame and vessel further comprises rotating the mount relative to the sliding arm.

18. The method of claim 13, wherein sliding the handle in a first direction such that the first and second locking rods slide within their respective channels in the first direction to lock the device to the port of the flexible bag is carried out by a single-handed operation.

19. The method of claim 13, further comprising sliding the handle in a second direction to unlock the device from the port.

20. The method of claim 15, further comprising using the camera to image an interior of the flexible bag during the bioprocessing operation.

21. The method of claim 14, wherein during the bioprocessing operation the flexible bag moves relative to the vessel, and the device moves in response to movement of the flexible bag, such that the device maintains alignment with the port during the bioprocessing operation.
